(19) United States
(12) Patent Application Publication
(10) Pub. No.: US 2026/0041697 A1
(43) Pub. Date: Feb. 12, 2026

(54) Formulation

(71) Applicant: Curaleaf, Inc., New York, NY (US)

(72) Inventors: Mikael Sodergren, London (GB); Juan Pablo Martinez, London (GB)

(21) Appl. No.: 18/826,505

(22) Filed: Sep. 6, 2024

(30) Foreign Application Priority Data
Aug. 8, 2024 (GB) 2411714.5

(51) Int. Cl. A61K 31/00; A61K 31/01; A61K 31/015; A61K 31/045; A61K 47/44; A61P 25/04

(52) U.S. Cl. CPC A61K 31/658; A61K 31/01; A61K 31/015; A61K 31/045; A61K 47/44; A61P 25/04

(57) ABSTRACT

There is disclosed a composition comprising delta-9-tetrahydrocannabinol, cannabidiol, cannabigerol and a terpene component.

### CLAIM OF PRIORITY

This application claims the benefit of UK patent application no. 2411714.5, filed on Aug. 8, 2024. The entire contents of the foregoing are hereby incorporated by reference.

### FIELD OF THE INVENTION

The present invention relates to a composition that has analgesic properties and methods of using the same for the treatment of neuropathic pain.

### BACKGROUND

Neuropathic pain is a type of pain defined by the International Association for the Study of Pain (IASP) as pain caused by a lesion or disease of the somatosensory nervous system. Neuropathic pain may be central, relating to the central nervous system (CNS) including the brain, brainstem or spinal cord, or peripheral, relating to the peripheral nervous system (PNS).

There are many possible causes of neuropathic pain. For example, neuropathic pain may occur due to trauma to the peripheral nervous system (PNS) or central nervous system (CNS) such as brain injury or spinal cord injury, stroke, infectious diseases, metabolic syndromes such as diabetes mellitus or multiple sclerosis and related neuroinflammatory conditions. Further, in some patients the cause of neuropathic pain may be unknown.

Due to the diverse origins of neuropathic pain, treating the underlying diseases or lesions is often difficult and as a result, the management of neuropathic pain can be challenging. Current available pharmacologic treatments for neuropathic pain include antidepressants (tricyclic anti-depressants (TCAs) and selective noradrenaline reuptake inhibitors (SNRIs)), anticonvulsants such as gabapentinoids (pregabalin, gabapentin), sodium channel blockers (lidocaine) and opioids. However, these medications can cause side effects and, particularly in the case of opioids, are unsuitable for long-term use because of addiction and tolerance issues. Thus, there is a continuing need for effective treatments of neuropathic pain.

Cannabis sativa, commonly known as marijuana, is a plant containing hundreds of different components, including cannabinoids and terpenes.

The two main cannabinoids found in Cannabis sativa, delta-9-tetrahydrocannabinol (THC) and cannabidiol (CBD) along with cannabigerol (CBG), have been widely studied for their analgesic effects and are reported to produce pain relief. However, THC is psychoactive and adverse cognitive side effects have been reported with higher concentrations of THC. As a result, the use of higher concentrations of THC in analgesic medicinal preparations is limited, decreasing their effectiveness at treating neuropathic pain.

There is, therefore, a continuing need for effective treatments of neuropathic pain. In particular, the applicants have recognised the need for compositions which provide therapeutically effective amounts of cannabinoids to the patient, whilst limiting adverse side effects associated with higher concentrations of THC.

### SUMMARY OF THE INVENTION

In a first aspect of the present invention there is provided a composition comprising delta-9-tetrahydrocannabinol, cannabidiol, cannabigerol and a terpene component.

Delta-9-tetrahydrocannabinol (THC) is a cannabinoid that has the following formula (Formula 1):

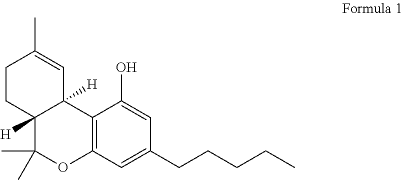 [SMILES: CCCCCc1cc(O)c2c(c1)OC(C)(C)[C@@H]1CCC(C)=C[C@@H]21]

Cannabidiol (CBD) is a cannabinoid that has the following formula (Formula 2):

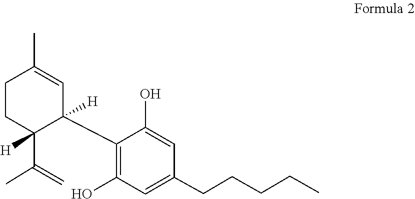 [SMILES: C=C(C)[C@@H]1CCC(C)=C[C@H]1c1c(O)cc(CCCCC)cc1O]

Cannabigerol (CBG) is a cannabinoid that has the following formula (Formula 3):

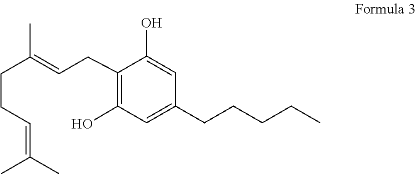 [SMILES: CCCCCc1cc(O)c(C/C=C(\C)CCC=C(C)C)c(O)c1]

Terpenes are a group of organic compounds derived from isoprene monomeric units. Terpenoids are derivatives of terpenes which usually contain oxygen atoms. Examples of terpenes and terpenoids include nerolidol (Formula 4), α-phellandrene (Formula 5), β-phellandrene (Formula 6), α-pinene (Formula 7), β-pinene (Formula 8), α-terpinene (Formula 9), β-terpinene (Formula 10), γ-terpinene (Formula 11), α-terpinene or terpinolene (Formula 12), borneol (Formula 13), limonene (Formula 14), β-caryophyllene (Formula 15), phytol (Formula 16), myrcene (Formula 17), α-terpineol (Formula 18), β-terpineol (Formula 19), γ-terpineol (Formula 20) and terpinene-4-ol (Formula 21):

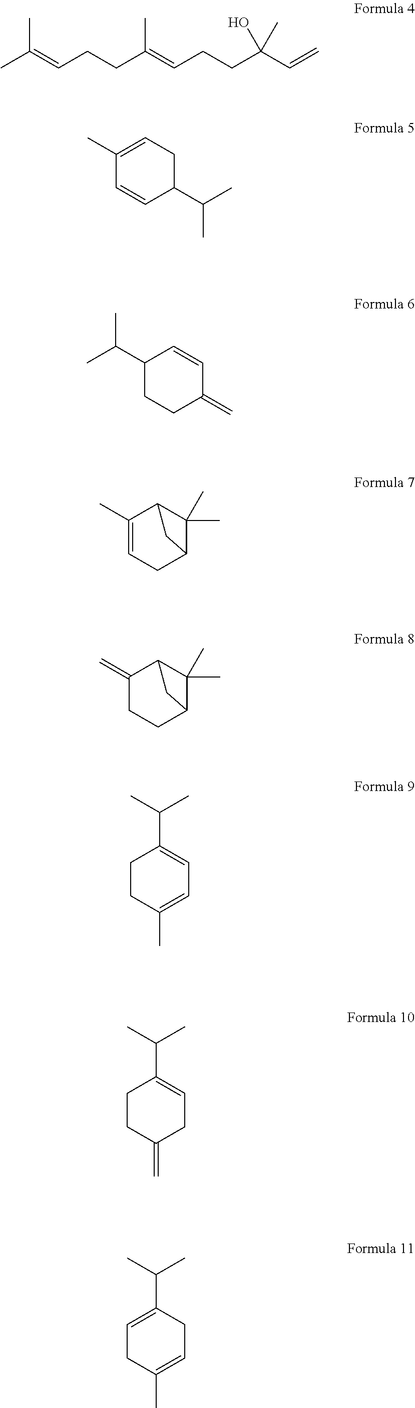

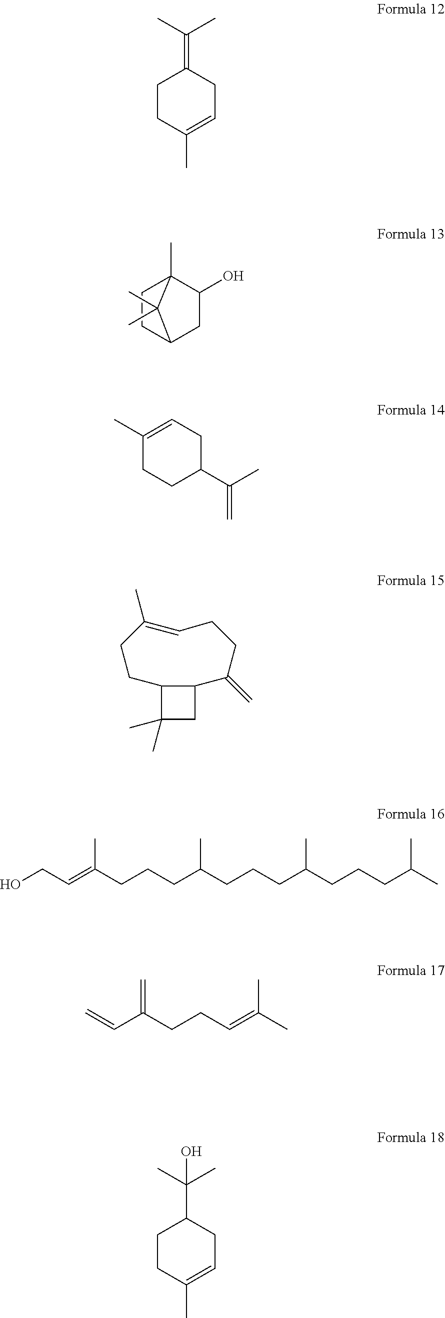

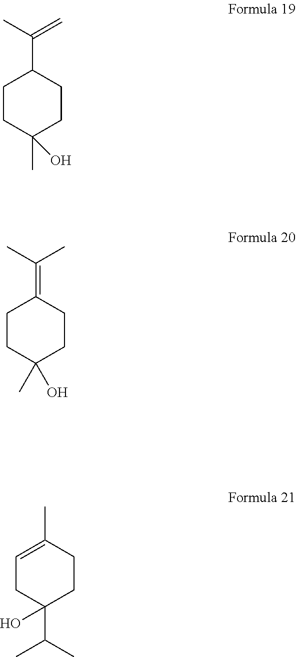

α-phellandrene may be in the form of (−)-α-phellandrene (Formula 5a) and (+)-α-phellandrene (Formula 5b):

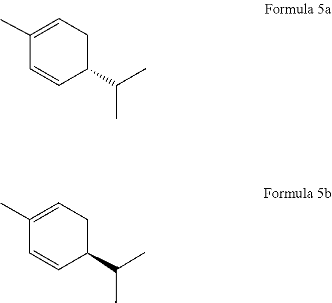

β-phellandrene may be in the form of (−)-β-phellandrene (Formula 6a) and (+)-β-phellandrene (Formula 6b):

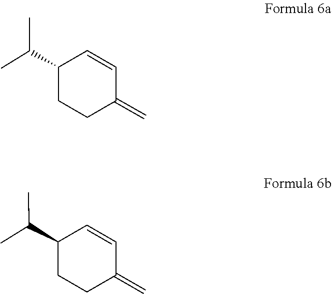

α-pinene may be in the form of (−)-α-pinene (Formula 7a) and (+)-α-pinene (Formula 7b):

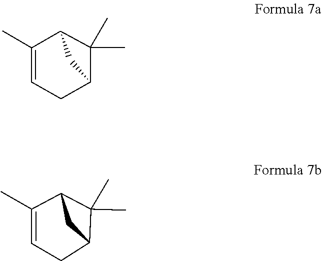

β-pinene may be in the form of (−)-β-pinene (Formula 8a) and (+)-3-pinene (Formula 8b):

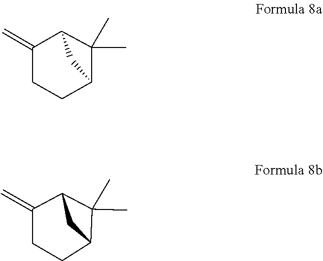

Borneol may be in the form of (−)-borneol (Formula 13a) and (+)-borneol (Formula 13b):

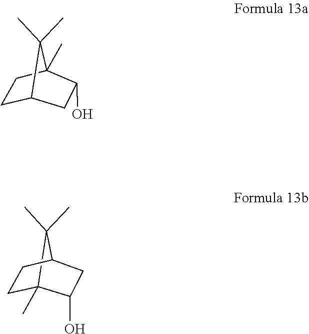

Limonene may be in the form of (−)-limonene (Formula 14a) and (+)-limonene (Formula 14b):

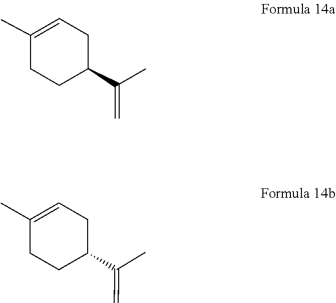

α-terpineol may be in the form of (−)-α-terpineol (Formula 18a) and (+)-α-terpineol (Formula 18b):

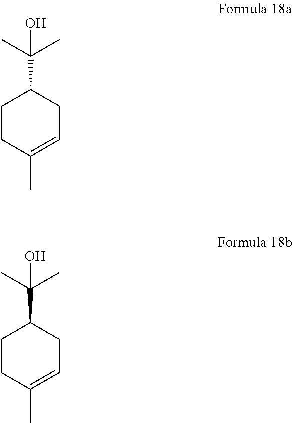

terpinene-4-ol may be in the form of (−)-terpinene-4-ol (Formula 21a) and (+)-terpinene-4-ol (Formula 21b):

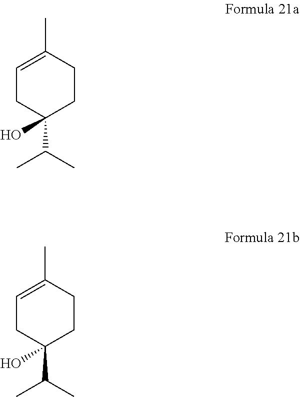

In this specification, δ-terpinene is also referred to as terpinolene.

The term “terpene component” is used to describe a combination of terpenes and/or terpenoid compounds.

The terpene component may comprise one or more terpenes, for example two or more terpenes, for example three or more terpenes, for example four or more terpenes, for example five or more terpenes.

The applicant has found that compositions comprising delta-9-tetrahydrocannabinol, cannabidiol, cannabigerol and a terpene component have analgesic properties and are effective at treating neuropathic pain. In particular, the applicant has found that the compositions described herein provide a synergistic effect, compared with the effects of the cannabinoids (delta-9-tetrahydrocannabinol, cannabidiol and cannabigerol individually or in combination) or terpenes (individually or in combination) by themselves.

In this specification, the combination of delta-9-tetrahydrocannabinol, cannabidiol and cannabigerol may be referred to as CBG+THC+CBD. In this specification, the combination of delta-9-tetrahydrocannabinol, cannabidiol, cannabigerol and a terpene component may be referred to as CBG+THC+CBD+ terpene mixture.

In this specification, the phrase “active ingredients” may be used to refer to the components of the composition that are pharmaceutically active and elicit a biological response. For example, delta-9-tetrahydrocannabinol, cannabidiol, cannabigerol and the terpene component may be collectively referred to as active ingredients.

Preferably, the composition is a liquid composition. Preferably, the delta-9-tetrahydrocannabinol, cannabidiol, cannabigerol and the terpene component are dissolved in a liquid (e.g., a carrier) to provide a liquid composition.

The delta-9-tetrahydrocannabinol may be derived from Cannabis sativa (e.g., extracted from Cannabis sativa). The cannabidiol may be derived from Cannabis sativa (e.g., extracted from Cannabis sativa). The cannabigerol may be derived from Cannabis sativa (e.g., extracted from Cannabis sativa). The terpene component may be derived from Cannabis sativa (e.g., extracted from Cannabis sativa). Alternatively, the terpene component may be derived from a plant other than Cannabis sativa (e.g., extracted from a plant other than Cannabis sativa) or synthesised chemically.

The terpene component may comprise from two to fifteen terpenes, for example three to twelve terpenes, for example four to ten terpenes, for example five to eight terpenes.

The terpenes may be selected from the group comprising nerolidol, α-phellandrene, α-pinene, δ-terpinene (terpinolene), borneol, limonene, β-caryophyllene, phytol, myrcene, α-terpineol, β-terpineol, γ-terpineol and terpinene-4-ol. Preferably, the terpenes are nerolidol, α-phellandrene, α-pinene, δ-terpinene (terpinolene) and β-caryophyllene. The terpenes may be derived from Cannabis sativa, for example extracted from Cannabis sativa. Alternatively, the terpenes may be derived from a plant other than Cannabis sativa (e.g., extracted from a plant other than Cannabis sativa) or synthesised chemically.

The terpene component may comprise nerolidol, α-phellandrene, α-pinene, δ-terpinene (terpinolene) and β-caryophyllene.

The amount of nerolidol may be from about 25% to about 30% by weight of the total amount of the terpene component (% w/w). The amount of α-phellandrene may be from about 5% to about 10% by weight of the total amount of the terpene component (% w/w). The amount of α-pinene may be from about 5% to about 10% by weight of the total amount of the terpene component (% w/w). The amount of δ-terpinene (terpinolene) may be from about 5% to about 10% by weight of the total amount of the terpene component (% w/w). The amount of β-caryophyllene may be from about 20% to about 25% by weight of the total amount of the terpene component (% w/w).

The applicant has found that compositions comprising delta-9-tetrahydrocannabinol, cannabidiol, cannabigerol and a terpene component comprising nerolidol, α-phellandrene, α-pinene, δ-terpinene (terpinolene) and β-caryophyllene have analgesic properties and are particularly effective at treating neuropathic pain.

The amount of delta-9-tetrahydrocannabinol may be from about 20% to about 40% by weight, for example from about 22% to about 38% by weight, for example from about 25% to about 35% by weight, for example from about 27% to about 33% by weight, for example from about 28% to about 32% by weight, for example from about 29% to about 31% by weight of the total amount of delta-9-tetrahydrocannabinol, cannabidiol, cannabigerol and the terpene component (% w/w).

The amount of delta-9-tetrahydrocannabinol may be less than 40% by weight, for example, less than 38% by weight, for example less than 36% by weight, for example less than 34% by weight, for example less than 32% by weight, for example less than 30% by weight of the total amount of delta-9-tetrahydrocannabinol, cannabidiol, cannabigerol and the terpene component (% w/w).

The amount of delta-9-tetrahydrocannabinol may be greater than 20% and less than 40% by weight of the total amount of delta-9-tetrahydrocannabinol, cannabidiol, cannabigerol and the terpene component (% w/w).

The amount of cannabidiol may be from about 20% to about 40% by weight, for example from about 22% to about 38% by weight, for example from about 25% to about 35% by weight, for example from about 27% to about 33% by weight, for example from about 28% to about 32% by weight, for example from about 29% to about 31% by weight of the total amount of delta-9-tetrahydrocannabinol, cannabidiol, cannabigerol and the terpene component (% w/w).

The amount of cannabigerol may be greater than 20% by weight, for example greater than 22% by weight, for example greater than 25% by weight, for example greater than 28% by weight, for example greater than 30% by weight of the total amount of delta-9-tetrahydrocannabinol, cannabidiol, cannabigerol and the terpene component (% w/w).

The amount of cannabigerol by weight of the total amount of delta-9-tetrahydrocannabinol, cannabidiol, cannabigerol and the terpene component in the composition may be greater than the amount of cannabigerol by weight of the total amount of delta-9-tetrahydrocannabinol, cannabidiol, cannabigerol and terpenes in Cannabis sativa (i.e., compositions or extracts derived directly from or extracted from Cannabis sativa) (% w/w).

The applicant has found that compositions comprising cannabigerol in an amount greater than 20% by weight of the total amount of delta-9-tetrahydrocannabinol, cannabidiol, cannabigerol and the terpene component (i.e., in an amount greater than in Cannabis sativa) (% w/w) are particularly effective at treating neuropathic pain.

The amount of cannabigerol may be from about 20% to about 40% by weight, for example from about 22% to about 38% by weight, for example from about 25% to about 35% by weight, for example from about 27% to about 33% by weight, for example from about 28% to about 32% by weight, for example from about 29% to about 31% by weight of the total amount of delta-9-tetrahydrocannabinol, cannabidiol, cannabigerol and the terpene component (% w/w).

The amount of terpene component may be greater than 5% by weight, for example greater than 10% by weight, for example greater than 15% by weight of the total amount of delta-9-tetrahydrocannabinol, cannabidiol, cannabigerol and the terpene component (% w/w).

The amount of terpene component by weight of the total amount of delta-9-tetrahydrocannabinol, cannabidiol, cannabigerol and the terpene component in the composition may be greater than the amount of terpenes by weight of the total amount of delta-9-tetrahydrocannabinol, cannabidiol, cannabigerol and terpenes in Cannabis sativa (i.e., compositions or extracts derived directly from or extracted from Cannabis sativa) (% w/w).

The applicant has found that compositions comprising a terpene component in an amount greater than 5% by weight of the total amount of delta-9-tetrahydrocannabinol, cannabidiol, cannabigerol and the terpene component (i.e., in an amount greater than in Cannabis sativa) (% w/w) are particularly effective at treating neuropathic pain.

The amount of terpene component may be from about 5% to 15% by weight, for example from about 6% to about 14% by weight, for example from about 7% to about 13% by weight, for example from about 8% to about 12% by weight, for example from about 9% to about 11% by weight of the total amount of delta-9-tetrahydrocannabinol, cannabidiol, cannabigerol and the terpene component (% w/w).

The composition may comprise each of the delta-9-tetrahydrocannabinol, cannabidiol, cannabigerol and a terpene component in any of the amounts set out above.

Preferably, the composition comprises delta-9-tetrahydrocannabinol in an amount from about 20% to about 40% by weight, for example from about 22% to about 38% by weight, for example from about 25% to about 35% by weight, for example from about 27% to about 33% by weight, for example from about 28% to about 32% by weight, for example from about 29% to about 31% by weight of the total amount of delta-9-tetrahydrocannabinol, cannabidiol, cannabigerol and the terpene component (% w/w); cannabidiol in an amount from about 20% to about 40% by weight, for example from about 22% to about 38% by weight, for example from about 25% to about 35% by weight, for example from about 27% to about 33% by weight, for example from about 28% to about 32% by weight, for example from about 29% to about 31% by weight of the total amount of delta-9-tetrahydrocannabinol, cannabidiol, cannabigerol and the terpene component (% w/w); cannabigerol in an amount from about 20% to about 40% by weight, for example from about 22% to about 38% by weight, for example from about 25% to about 35% by weight, for example from about 27% to about 33% by weight, for example from about 28% to about 32% by weight, for example from about 29% to about 31% by weight of the total amount of delta-9-tetrahydrocannabinol, cannabidiol, cannabigerol and the terpene component (% w/w); and a terpene component in an amount from about 5% to 15% by weight, for example from about 6% to about 14% by weight, for example from about 7% to about 13% by weight, for example from about 8% to about 12% by weight, for example from about 9% to about 11% by weight of the total amount of delta-9-tetrahydrocannabinol, cannabidiol, cannabigerol and the terpene component (% w/w).

Preferably, the composition comprises delta-9-tetrahydrocannabinol in an amount from about 20% to about 40% by weight of the total amount of delta-9-tetrahydrocannabinol, cannabidiol, cannabigerol and the terpene component (% w/w); cannabidiol in an amount from about 20% to about 40% by weight of the total amount of delta-9-tetrahydrocannabinol, cannabidiol, cannabigerol and the terpene component (% w/w); cannabigerol in an amount from about 20% to about 40% by weight of the total amount of delta-9-tetrahydrocannabinol, cannabidiol, cannabigerol and the terpene component (% w/w); and a terpene component in an amount from about 5% to 15% by weight of the total amount of delta-9-tetrahydrocannabinol, cannabidiol, cannabigerol and the terpene component (% w/w).

The ratio of delta-9-tetrahydrocannabinol:cannabidiol:cannabigerol may be X:Y:Z by weight, wherein X, Y and Z are each independently from 0.5 to 1:5. Preferably, the ratio of delta-9-tetrahydrocannabinol:cannabidiol:cannabigerol is about 1:1:1 by weight.

The ratio of cannabigerol to delta-9-tetrahydrocannabinol:cannabidiol may be higher than the ratio of cannabigerol to delta-9-tetrahydrocannabinol:cannabidiol in Cannabis sativa (i.e., compositions or extracts derived directly from or extracted from Cannabis sativa).

The ratio of delta-9-tetrahydrocannabinol:cannabidiol:cannabigerol: the terpene component may be from about 3:3:3:1 to about 4:4:4:1 by weight.

The ratio of the terpene component to delta-9-tetrahydrocannabinol:cannabidiol:cannabigerol may be higher than the ratio of the terpenes to delta-9-tetrahydrocannabinol:cannabidiol:cannabigerol in Cannabis sativa (i.e., compositions or extracts derived directly from or extracted from Cannabis sativa).

The ratio of cannabigerol to delta-9-tetrahydrocannabinol:cannabidiol:terpene component may be higher than the ratio of cannabigerol to delta-9-tetrahydrocannabinol:cannabidiol:terpene component in Cannabis sativa (i.e., compositions or extracts derived directly from or extracted from Cannabis sativa).

The composition may further comprise a carrier. The carrier may function to aid the administration of the composition and may also function to stabilise or preserve the active ingredients without interacting with the active ingredients. Preferably, the carrier comprises a carrier oil. Preferably, the carrier oil comprises medium-chain triglyceride (MCT) oil. MCT oil is made up of medium-chain triglycerides, and generally includes triglycerides having chains of carbon molecules ranging from 6 to 12 carbon atoms in length.

The total amount of delta-9-tetrahydrocannabinol may be from about 1% to about 3% by weight of the volume of the composition (% w/v). The total amount of cannabidiol may be from about 1% to about 3% by weight of the volume of the composition (% w/v). The total amount of cannabigerol may be from about 1% to about 3% by weight of the volume of the composition (% w/v). The total amount of terpene component may be from about 0.5% to about 2% by weight of the volume of the composition (% w/v).

The total amount of delta-9-tetrahydrocannabinol, cannabidiol, cannabigerol and the terpene component may be from about 5% to about 10% by weight, for example from about 6% to about 9% by weight, for example from about 7% to about 9% by weight of the volume of the composition (% w/v).

Preferably, the composition is a pharmaceutical composition, for example a human pharmaceutical composition. Preferably, the composition is in the form of an oil.

The composition may be in a form suitable for one or more of oral, transdermal (e.g., topical), transmucosal (e.g., sublingual, e.g., buccal) or nasal administration or inhalation. Preferably, the composition may be in a form suitable for sublingual or topical administration or inhalation.

The composition may reduce or inhibit capsaicin responses in DRG neurons compared to baseline/control. The composition may inhibit capsaicin responses in DRG neurons compared to baseline/control. The composition may desensitise TRPV1 in DRG neurons compared to baseline/control. The composition may reduce or inhibit calcium influx at TRPV1 channels compared to baseline/control.

In a further aspect of the present invention, there is provided a composition according to any statement set out above for use as a medicament.

In a further aspect of the present invention, there is provided a composition according to any statement set out above for use in the treatment of pain.

In a further aspect of the present invention, there is provided a composition according to any statement set out above for use in the treatment of neuropathic pain.

The composition for use in the treatment of pain (e.g., neuropathic pain) may be administered orally, transdermally (e.g., topically), transmucosally (e.g., sublingually, e.g., buccally) or by inhalation (e.g., nasal inhalation). Preferably, the composition may be administered sublingually, topically or by inhalation.

The composition for use in the treatment of pain (e.g., neuropathic pain) may be administered in a daily dose of about 8 mg to about 12 mg of THC, about 8 mg to about 12 mg of CBD, about 8 mg to about 12 mg of CGB and about 3 mg to about 5 mg of the terpene component.

In a further aspect of the present invention, there is provided a method of treating pain, comprising a step of administering to a subject a composition as set out above.

In a further aspect of the present invention, there is provided a method of treating neuropathic pain, comprising a step of administering to a subject a composition as set out above.

The method of treating pain (e.g., neuropathic pain) may comprise administering a daily dose of about 8 mg to about 12 mg of THC, about 8 mg to about 12 mg of CBD, about 8 mg to about 12 mg of CGB and about 3 mg to about 5 mg of the terpene component.

In a further aspect of the present invention, there is provided a composition according to any statement set out above for use in the manufacture of a medicament for the treatment of pain.

In a further aspect of the present invention, there is provided a composition according to any statement set out above for use in the manufacture of a medicament for the treatment of neuropathic pain.

### DETAILED DESCRIPTION

Specific embodiments of the invention will now be described by way of examples, with reference to the accompanying drawings, in which:

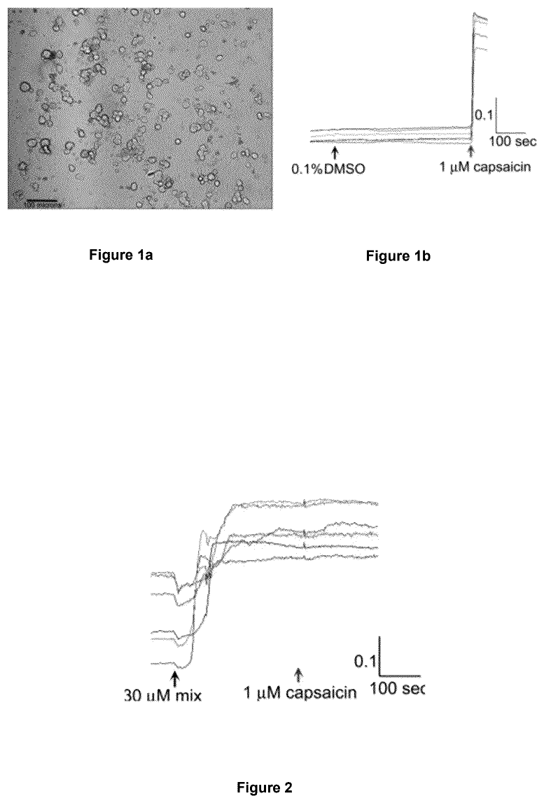

FIG. 1a shows a photomicrograph of adult rat DRG neurons, with individual cells highlighted for analysis (bar=100 μM), as described in comparative Example 1;

FIG. 1b shows the response of adult rat DRG neurons to vehicle control (0.1% DMSO) followed by 1 μM capsaicin in a functional calcium imaging assay, as described in comparative Example 1;

FIG. 2 shows the response of adult rat DRG neurons to a combination of CBG, THC and CBD in a 1:1:1 ratio at a concentration of 30 μM followed by 1 μM capsaicin in a functional calcium imaging assay, as described in comparative Example 1;

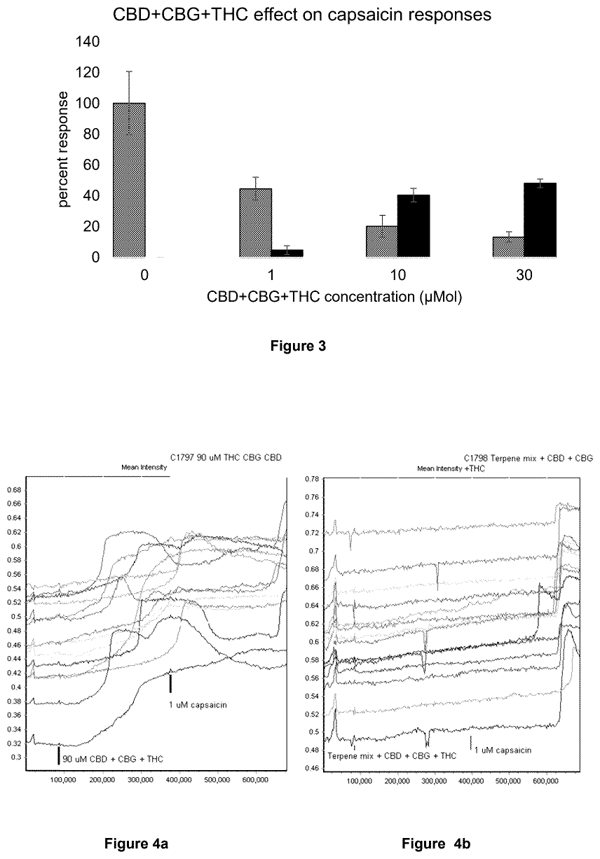

FIG. 3 shows the dose-related calcium response to the combination of CBG, THC and CBD in a 1:1:1 ratio at concentrations of 3 μM, 30 μM and 90 μM and the inhibitory effect of the combination of CBG, THC and CBD on capsaicin responses expressed as a percentage of calcium influx in response to 1 μM capsaicin, as described in comparative Example 1;

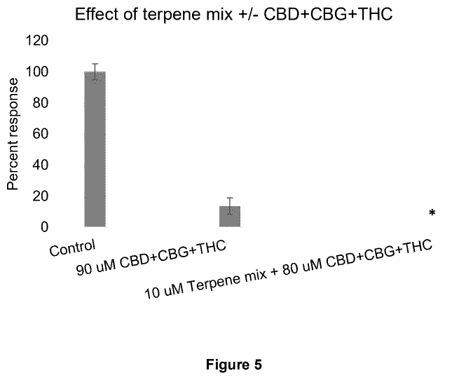

FIG. 4a shows the response of adult rat DRG neurons to a combination of CBG, THC and CBD in a 1:1:1 ratio at a concentration of 90 μM followed by 1 μM capsaicin in a functional calcium imaging assay, as described in Example 3;

FIG. 4b shows the response of adult rat DRG neurons to combination of CBG, THC and CBD in a 1:1:1 ratio at a concentration of 80 μM and terpenes at a concentration of 10 μM in a 1:1:1:1:1 ratio followed by 1 μM capsaicin in a functional calcium imaging assay, as described in Example 3;

FIG. 5 shows the inhibitory effect of neurons to a combination of CBG, THC, CBD and terpenes on capsaicin responses expressed as a percentage of calcium influx in response to 1 μM capsaicin, as described in Example 3;

### EXAMPLES

The following experiments were conducted to investigate the antinociceptive effect of cannabinoids CBD, CBG and THC and various terpenes in cultured rat sensory neurons, following activation of TRPV1 (transient receptor potential cation channel subfamily V member 1) in an established in vitro model of neuronal hypersensitivity (as described by Anand et al. (2020)).

TRPV1 is a non-selective cation channel expressed in small sensory neurons of the DRG with a high permeability to calcium (Ca²⁺). TRPV1 plays an important role in pain signalling, detecting noxious stimuli such as temperatures of 43° C. and above, inflammatory mediators, low pH and capsaicin. When DRG neurons are stimulated with capsaicin, TRPV1 is activated and a calcium (Ca²⁺) influx is generated. The effect of CBD, CBG and THC and various terpenes on TRPV1 followed by capsaicin activation is then determined in a functional calcium imaging assay, using Fura-2 pentakis (acetoxymethyl) ester (Fura 2-AM) indicator dye.

### Comparative Example 1: Effect of CBG, CBD and THC Combined on TRPV1 Activation by Capsaicin in Adult Rat DRG Neurons

### Materials and Methods

### Neuronal Cultures

DRG neurons were prepared using adult female Witsar rats, as described by Anand et al. (2021). Bilateral DRG were harvested in Ham's F12 medium under sterile conditions, and enzyme digested in 2 mL Ham's F12 medium containing collagenase (0.2%) and dispase (0.5%), at 37° C. for 3 hours. The enzyme digested tissue was triturated in 1 mL BSF2 medium containing 100 ng/ml nerve growth factor (NGF-7s, Merck Life Science, UK Ltd), and 50 ng/mL glial cell-line derived neurotrophic factor (GDNF, Merck Life Science, UK Ltd), trypsin inhibitor and DNase, to obtain a neuronal cell suspension. 8000-10000 neurons in 200 μL medium were plated onto each of 20 glass-bottom petri dishes (MatTek Corp, USA), coated with 20 μg/mL poly-l-lysine and 20 μg/mL laminin. The cultures were incubated at 37° C. for 45 minutes to allow the cells to attach before adding 2 mL BSF2 medium. 24 hours later 5 μM cytosine arabinoside was added to all dishes to inhibit the growth of non-neuronal cells.

### Calcium Imaging

Calcium imaging was performed between 2 and 4 days after plating the neurons, as described by Anand et al. (2021).

The culture medium was aspirated from each dish, and the neurons rinsed with 4-(2-Hydroxyethyl) piperazine-1-ethanesulfonic acid (HEPES) buffered Hank's Balanced salt solution (HBSS), containing 0.1% Bovine Serum Albumin (BSA) (pH 7.4). 1 mL HEPES buffered HBSS containing 2 μMol Fura-2 AM (Life Technologies, Paisley, UK), was added to each dish, and the petri dishes were incubated at 37° C. for 40 minutes. The medium was then replaced with HEPES-HBSS containing 0.1% BSA for 20 minutes to allow de-esterification in the dark.

A total of 12-15 healthy neurons were selected for each experiment (vehicle treated control and CBG+THC+CBD) using a 10× objective lens under brightfield illumination for each experiment, and a region of interest was highlighted in each neuron (see FIG. 1a). The neuron culture was alternately excited at 340 and 380 nm (λex) (510 λem) wavelengths for one minute to obtain the intracellular bound/unbound Ca²⁺ ratio. A stable baseline of the 340/380 nm ratio was recorded.

For the vehicle control, following baseline recording, 0.1% dimethysulfoxide (DMSO) was added to the dish, followed by 1 μM capsaicin 5 minutes later. In separate dishes, following baseline recording, a combination of CBG+THC+CBD (1:1:1 ratio, concentrations of 3 μM, 30 μM or 90 μM) were added at the indicated concentrations, followed by 1 μM capsaicin 5 minutes later.

One image was captured every two seconds in each of three channels: brightfield, 340 nm and 380 nm λex and the mean 340/380 nm λex ratio was recorded for each neuron to reveal intracellular Ca²⁺ changes due to capsaicin or cannabinoid application. The largest calcium responses were selected for analysis. The responses were recorded as the difference between baseline (mean 340/380 nm λex ratio) just before addition of the cannabinoid or capsaicin, and peak after the addition expressed as a percentage of the control obtained from the same animal specimen.

The response of adult rat DRG neurons to vehicle control (0.1% DMSO) followed by capsaicin is shown in FIG. 1b. The response of adult rat DRG neurons to 30 μM CBG+THC+CBD are shown in FIG. 2. In FIG. 1b and FIG. 2, the x-axis shows the time in seconds and the y-axis shows the intracellular 340/380 (bound/unbound calcium) ratio, in individual neurons depicted by different colours. Table 1 shows the dose related responses to combined CBG, CBD, and THC application, and corresponding reduction of capsaicin responses.

### Cannabinoid Solutions

Stock solution of CBG (from extract paste, #401100P Curaleaf International, London, UK), was prepared at 316 mM in DMSO. 100 mM CBD stock solution was prepared in DMSO (#200003, Curaleaf International, London, UK), and delta 9THC (#401100P, Curaleaf International, London, UK) was prepared in DMSO at 317 mM. All stock solutions were aliquoted, stored at −20° C., and freshly thawed prior to use. Intermediate dilutions were freshly prepared at 1000× final concentration, so that the final concentration of vehicle was 0.1%. THC, CBD and CBG were combined in 1:1:1 proportion by adding each cannabinoid at 1 mM, 10 mM or 30 mM to give a mixture containing 3 mM, 30 mM or 90 mM total in DMSO. This was diluted 1:1000 so that the final concentration of the added mixture was 3 μM, 30 μM or 90 μM for the combination. Capsaicin stock solution was prepared in ethanol as a 100 mM solution, aliquoted and stored at −20° C., until use; intermediate dilution of 500 UM was freshly prepared prior to use. All chemicals described above were obtained from Merck (Gillingham, UK), unless otherwise indicated.

### Data Analysis

The combined cannabinoid and capsaicin responses from 12 to 15 neurons were averaged for each concentration for each rat, and normalized to vehicle treated controls. The number of rats tested for each concentration, and the total number of neurons, from which the data is derived, is indicated in Table 1 below. Average values for the cannabinoid concentrations were compared to the control using a one-tailed Student's t-test. Pearson's correlation coefficient was used to determine a correlation between calcium influx in response to the cannabinoids, and calcium influx in response to capsaicin administration in the presence of the cannabinoids. All analyses were carried out using GraphPad Prism software. *P<0.05, **P<0.01, ***P<0.001.

### Results

In the presence of the vehicle control (0.1% DMSO), no change in baseline intracellular bound/unbound Ca²⁺ ratio was observed. Capsaicin sensitive neurons responded immediately, within seconds of application of 1 μM capsaicin, as an immediate increase in intracellular 340/380 ratio that was sustained for several minutes (see FIG. 1b).

As shown in FIG. 2, 30 μM of CBG+THC+CBD applied in a 1:1:1 ratio elicited dose-related calcium influx after a delay, resulting in desensitisation to capsaicin stimulation. While the application of 30 μM of CBG+THC+CBD caused neuronal activation, the calcium influx induced was less than that of the control (1 μM capsaicin).

The results in Table 1 show that CBG+THC+CBD, applied in a 1:1:1 ratio at 3 μM, 30 μM, and 90 μM, elicited dose-related calcium influx, with maximum influx at 90 μM. Capsaicin responses (expressed as a percentage of calcium influx in response to 1 μM capsaicin) were dose-dependently diminished at 3 μM, 30 μM, and 90 μM CBG+THC+CBD. There was a high correlation between cannabinoid-mediated calcium influx and reduction of capsaicin responses, Pearson's coefficient=−1.00.

TABLE 1
Effect of combined CBG + CBD + THC on adult
rat DRG neurons followed by 1 μM capsaicin

| | | | | |
|---|---|---|---|---|
| CBG + CBD + THC | 0 | 3 | 30 | 90 |
| concentration (μM) | | | | |
| CBG + CBD + THC | 0 | 4.6 ± | 40.3 ± | 48 ± |
| Response | | 2.8 | 4.3 | 2.8 |
| Mean ± s.e.m | | | | |
| N (neurons) | 8(52) | 6(47) | 6(57) | 6(55) |
| Capsaicin response | 100 ± | 44.4 ± | 20.1 ± | 13.0 ± |
| Mean ± s.e.m | 20.5 | 7.5 | 7.1 ** | 3.4 ** |
| N (neurons) | 8(52) | 6(47) | 6(62) | 6(55) |

The results demonstrate that CBG+THC+CBD applied in a 1:1:1 ratio at 3 μM, 30 μM, and 90 μM inhibit neuronal hypersensitivity in cultured rat sensory neurons, following activation of TRPV1. These results highlight the potential anti-nociceptive application of CBG, THC and CBD in combination.

### Comparative Example 2: Effect of Terpenes on TRPV1 Activation by Capsaicin in Adult Rat DRG Neurons

### Materials and Methods

### Neuronal Cultures

Adult rat DRG neurons were prepared as described in comparative Example 1.

### Calcium Imaging

Calcium imaging was performed as described in comparative Example 1.

For the vehicle control, 0.1% ethanol was added to the dish, followed by 1 μM capsaicin 5 minutes later. In separate dishes, following baseline recording, individual terpenes (borneol, phytol, terpenolene, α-pinene, (+)-limonene, α-phellandrene, myrcene, β-caryophyllene, nerolidol and α-terpineol) were added at either 0.001 μM, 0.01 μM, 0.1 μM, 1 μM, 10 μM or 100 μM, followed 1 μM capsaicin 5 minutes later.

The percentage of adult rat DRG neurons with delayed calcium increases (i.e., 6 to 8 minutes) to the individual terpenes followed by 1 μM capsaicin are shown in Table 2.

### Terpene Solutions

The terpenes borneol, phytol, terpenolene, α-pinene, (+)-limonene, α-phellandrene, myrcene, β-caryophyllene, nerolidol and α-terpineol were freshly prepared prior to use at 1000× final concentration in ethanol. Aliquots of capsaicin stock solution (100 mM) prepared in ethanol were frozen at −20° C. and used for preparing intermediate stock solution of 500 UM prior to use. All chemicals described above were obtained from Merck (Gillingham, UK), unless indicated otherwise.

### Results

In the presence of the vehicle control (0.1% ethanol), no change in baseline intracellular bound/unbound Ca²⁺ ratio was observed. Capsaicin sensitive neurons responded immediately, within seconds of application of 1 μM capsaicin, as an immediate increase in intracellular 340/380 ratio that was sustained for several minutes.

TABLE 2
Percentage of rat DRG neurons with delayed calcium increases
(i.e., 6 to 8 minutes) to individual terpenes followed
by 1 μM capsaicin (data from n = 3 rats/terpene).

| | Concentration | | | | | |
|---|---|---|---|---|---|---|
| | μM | | | | | |
| | 0.001 | 0.01 | 0.1 | 1 | 10 | 100 |
| Borneol | 21 | 0 | 0 | 27 | 33 | 66 |
| Caryophyllene | 50 | 0 | 66 | 66 | 66 | 95 |
| Limonene | 66 | 66 | 16 | 39 | 34 | 70 |
| Phellandrene | 36 | 66 | 71 | 93 | 66 | 2 |
| Phytol | 0 | 49 | 49 | 68 | 66 | 68 |
| Pinene | 100 | 50 | 66 | 16 | 75 | 33 |
| Nerolidol | 68.8 | 33 | 33 | 100 | 97.7 | 100 |
| Myrcene | 0 | 44 | 44 | 66 | 66 | 66 |
| Terpinolene | 20 | 46 | 70 | 100 | 71 | 19 |
| Terpineol | 66.6 | 80.3 | 100 | 68.7 | 100 | 93 |

The results in Table 2 show that for various concentrations of terpinolene, α-pinene, α-phellandrene, β-caryophyllene, nerolidol and α-terpineol, capsaicin responses were completely inhibited for 6-8 minutes, in the majority of capsaicin sensitive neurons. Most neurons treated with terpenes responded to capsaicin after 6-8 minutes. Without wanting to be bound by theory, it is believed that the delayed responses to capsaicin (i.e., 6-8 minutes) were due to calcium release from the endoplasmic reticulum, not as a result of calcium influx. Therefore, calcium influx was completely blocked in the presence of terpenes in most capsaicin sensitive neurons.

The results in Table 2 demonstrate that terpenes ranging in concentration from 0.001 to 100 μM have potent inhibitory effects in DRG neurons, by blocking calcium influx in response to capsaicin activation of TRPV1. These results highlight the potential anti-nociceptive application of terpenes.

### Example 3: Combined Effect of CBG, CBD, THC and Terpenes on TRPV1 Activation by Capsaicin in Adult Rat DRG Neurons

The terpenes investigated were a mixture of nerolidol, α-phellandrene, α-pinene, terpinolene and β-caryophyllene in a 1:1:1:1:1 ratio.

### Materials and Methods

### Neuronal Cultures

DRG neurons were prepared as described in comparative Example 1.

### Calcium Imaging

Calcium imaging was performed as described in comparative Example 1.

For the vehicle control, following baseline recording, 0.1% ethanol was added to the dish, followed by 1 μM capsaicin 5 minutes later.

In separate dishes, following baseline recording, a combination of CBG+THC+CBD (1:1:1 ratio, concentration of 90 M) and a combination of CBG+THC+CBD (1:1:1 ratio, concentration of 80 μM)+a terpene mixture (nerolidol, α-phellandrene, α-pinene, terpinolene and β-caryophyllene, in a 1:1:1:1:1 ratio, concentration of 10 μM) were added, followed by 1 μM capsaicin 5 minutes later.

The response of adult rat DRG neurons to 90 μM CBG+THC+CBD is shown in FIG. 4a. The response of adult rat DRG neurons to 80 μM CBG+THC+CBD+10 μM terpene mixture is shown in FIG. 4b. In FIG. 4a and FIG. 4b, the x-axis shows the time in seconds and the y-axis shows the intracellular 340/380 (bound/unbound calcium) ratio, in individual neurons depicted by different colours. FIG. 5 shows the inhibitory effect of 90 μM CBG+THC+CBD and 80 μM CBG+THC+CBD+10 μM terpene mixture on capsaicin responses expressed as a percentage of calcium influx in response to 1 μM capsaicin.

Table 3 shows normalized values of capsaicin response amplitudes, following treatment with control (0.1% ethanol), CBG+CBD+THC and CBG+CBD+THC+ terpene mixture.

### Cannabinoid and Terpene Solutions

The cannabinoid and terpene solutions were prepared as described in comparative Examples 1 and 2.

### Data Analysis

Data analysis was performed as described in comparative Example 1.

### Results

Adult rat DRG neurons treated with 90 μM CBD+CBG+THC in a 1:1:1 ratio, activated calcium influx and inhibited capsaicin responses to 18±6.9% of vehicle treated controls. It was found that in the added presence of 10 μM terpene mixture, capsaicin responses were inhibited to 0.6±0.6% of control.

Capsaicin response inhibition due to the combination of CBD, CBG and THC was significantly enhanced in the added presence of the terpene mixture. This further inhibition due to the combined CBD, CBG and THC and terpene mixture, was significantly greater than the effect of the cannabinoid mixture alone (*P<0.05 T-test).

TABLE 3
Effect of control, combined CBG + CBD + THC and
combined CBG + CBD + THC + terpene mixture on adult
rat DRG neurons followed by 1 μM capsaicin

| | Control | | 10 μM terpene |
|---|---|---|---|
| | (0.1% | 90 μM | mix + 80 μM |
| | Ethanol) | CBD + CBG + THC | CBD + CBG + THC |
| R1 | 95.74468 | 30 | 0 |
| R2 | 108.5106 | 6 | 1.8 |
| R3 | 95.74468 | 18.2 | 0 |
| Mean | 100 | 18.06667 | 0.6 |
| Standard | 7.370429 | 12.00056 | 1.03923 |
| Deviation | | | |
| n | 3 | 3 | 3 |
| s.e.m. | 4.255319 | 6.928524 | 0.6 |
| T test | | 0.032973 | |

These data show that the cannabinoid mixture comprising of 90 μM CBG+CBD+THC had a potent inhibitory effect on capsaicin responses. It was found that the addition of 10 μM of a terpene mixture combined with 80 μM of the cannabinoid mixture completely inhibited capsaicin responses to 0.6±0.6% of control.

Without wanting to be bound by theory, it is believed that mechanism of terpene mediated TRPV1 inhibition is different to that of the cannabinoids THC, CBD, and CBG. Therefore, the combinations of terpenes and the cannabinoids THC, CBD, and CBG show enhanced effects, via additive or synergistic effects involving different signalling pathways.

### Example 4: Composition 1

Delta-9-tetrahydrocannabinol
Cannabidiol
Cannabigerol
Nerolidol
α-Phellandrene
α-Pinene
Terpinolene
β-Caryophyllene
MCT oil

The amount of delta-9-tetrahydrocannabinol is 30% by weight of the total amount of delta-9-tetrahydrocannabinol, cannabidiol, cannabigerol and the terpene component. The amount of cannabidiol is 30% by weight of the total amount of delta-9-tetrahydrocannabinol, cannabidiol, cannabigerol and the terpene component. The amount of cannabigerol is 30% by weight of the total amount of delta-9-tetrahydrocannabinol, cannabidiol, cannabigerol and the terpene component. The amount of the terpene component is 10% by weight of the total amount of delta-9-tetrahydrocannabinol, cannabidiol, cannabigerol and the terpene component. Specifically, the amount of α-pinene is about 1.6%, the amount of α-phellandrene is about 1.6%, the amount of terpinolene is about 1.6%, the amount of nerolidol is about 2.7% and the amount of β-caryophyllene is about 2.4% by total weight of the amount of delta-9-tetrahydrocannabinol, cannabidiol, cannabigerol and the terpene component.

The total amount of delta-9-tetrahydrocannabinol, cannabidiol, cannabigerol and the terpene component is about 7% by weight of the total composition.

### Example 5: Manufacture of Composition 1

The components that make up composition 1 were obtained from a commercial supplier. The general process for extracting THC, CBD and CBG from Cannabis sativa flowers uses ethanol. The extracts undergo steps of decarboxylation and winterisation. The ethanol is then removed by distillation and MCT oil added.

15% THC extract, comprising delta-9-tetrahydrocannabinol and MCT oil, 15% CBD extract comprising cannabidiol and MCT oil, 5% CBG extract comprising cannabigerol and MCT oil and a terpene blend comprising nerolidol, α-phellandrene, α-pinene, terpinolene, β-caryophyllene and MCT oil were mixed. MCT oil was then added to the mixture to form the composition.

Table 4 shows the volume of each component required to produce 10 mL of composition 1 and the percentage ratio of API in the composition.

TABLE 4
The volume of each component required to
produce 10 mL of composition 1 and the percentage
ratio of API in the composition.

| | | Ratio in | API | API Ratio in |
|---|---|---|---|---|
| | Volume | formulation | Weight | formulation |
| Component | (mL) | % (V/V) | (mg) | % (W/V) |
| ET15 (THC API | 1.33 | 13.33 | 200 | 2% |
| Extract) | | | | |
| EC15 (CBD API | 1.33 | 13.33 | 200 | 2% |
| Extract) | | | | |
| CBG 5% (CBG API | 4.0 | 40 | 200 | 2% |
| Extract) | | | | |
| Terpenes | 0.066 | 0.66 | 66.42 | 0.66% |
| MCT Oil | 3.267 | 32.67 | — | 93.34% |

Table 5 shows the weight of each terpene in 10 mL of composition 1.

TABLE 5
The weight of each terpene in 10 mL of composition 1

| | API Weight |
|---|---|
| Terpene | (mg) |
| Nerolidol | 17.68 |
| α-Phellandrene | 10.83 |
| α-Pinene | 10.83 |
| Terpinolene | 10.83 |
| β-Caryophyllene | 16.25 |

1. A composition comprising:
(i) delta-9-tetrahydrocannabinol;
(ii) cannabidiol;
(iii) cannabigerol; and
(iv) a terpene component.

2. The composition of claim 1 wherein the composition is a liquid composition.

3. The composition of claim 1 wherein the terpene component comprises one or more terpenes.

4. The composition of according to claim 3 wherein the terpene(s) are selected from nerolidol, α-phellandrene, α-pinene, δ-terpinene (terpinolene), borneol, limonene, β-caryophyllene, phytol, myrcene, α-terpineol, β-terpineol, γ-terpineol and terpinene-4-ol.

5. The composition according to claim 1, wherein the delta-9-tetrahydrocannabinol is present in an amount from about 20% to about 40% by weight of the total amount of delta-9-tetrahydrocannabinol, cannabidiol, cannabigerol and the terpene component.

6. The composition according to claim 1, wherein the cannabidiol is present in an amount from about 20% to about 40% by weight of the total amount of delta-9-tetrahydrocannabinol, cannabidiol, cannabigerol and the terpene component.

7. The composition according to claim 1, wherein the cannabigerol is present in an amount from about 20% to about 40% by weight of the total amount of delta-9-tetrahydrocannabinol, cannabidiol, cannabigerol and the terpene component.

8. The composition according to claim 1, wherein the terpene component is present in an amount greater than 5% by weight of the total amount of delta-9-tetrahydrocannabinol, cannabidiol, cannabigerol and the terpene component.

9. The composition according to claim 1, wherein the terpene component is present in an amount from about 5% to about 15% by weight of the total amount of delta-9-tetrahydrocannabinol, cannabidiol, cannabigerol and the terpene component.

10. The composition according to claim 1, wherein the ratio of delta-9-tetrahydrocannabinol:cannabidiol:cannabigerol is X:Y:Z by weight, wherein X, Y and Z are each independently from 0.5 to 1:5.

11. The composition according to claim 1, wherein the ratio of delta-9-tetrahydrocannabinol:cannabidiol:cannabigerol: the terpene component is from 3:3:3:1 to 4:4:4:1 by weight.

12. The composition according to claim 1, further comprising a carrier.

13. The composition according to claim 12, wherein the carrier comprises a carrier oil.

14. The composition according to claim 13, wherein the carrier oil comprises medium-chain triglyceride (MCT) oil.

15. The composition according to claim 1 wherein the total amount of delta-9-tetrahydrocannabinol, cannabidiol, cannabigerol and the terpene component is from about 5% to about 10% by weight of the composition.

16. The composition according to claim 1, wherein the composition is a pharmaceutical composition.

17. The composition according to claim 1, wherein the composition is in a form suitable for one or more of oral, transdermal (e.g., topical), transmucosal (e.g., sublingual, e.g., buccal) or nasal administration or inhalation.

18. The composition according to claim 1, wherein when administered to a subject the composition reduces or inhibits capsaicin responses in DRG neurons compared to baseline/control.

19. The composition according to claim 1, wherein when administered to a subject the composition desensitises TRPV1 in DRG neurons compared to baseline/control.

20. The composition according to claim 1, wherein when administered to a subject the composition reduces or inhibits calcium influx at TRPV1 channels compared to baseline/control.

21. (canceled)

22. (canceled)

23. (canceled)

24. A method of treating neuropathic pain comprising a step of administering to a subject a composition according to claim 1.

25. (canceled)

26. The method of claim 24, wherein the composition is administered orally, transdermally, transmucosally or by inhalation.
